(19) United States
(12) Patent Application Publication
(10) Pub. No.: US 2012/0004661 A1
(43) Pub. Date: Jan. 5, 2012

(54) ORTHOPEDIC BONE DRILL

(76) Inventor: John Leppelmeier, Bryan, OH (US)

(21) Appl. No.: 13/171,920

(22) Filed: Jun. 29, 2011

Related U.S. Application Data
(60) Provisional application No. 61/359,996, filed on Jun. 30, 2010.

(51) Int. Cl. A61B 17/16

(52) U.S. Cl. 606/80

(57) ABSTRACT

A drill bit includes a body extending along an axis and including a point end and a shank end. The drill bit also includes a first helical portion, having a first helix angle relative to the axis. The drill bit also includes a second helical portion, having a second helix angle relative to the axis which is different from the first helix angle.

### CROSS-REFERENCE TO RELATED APPLICATIONS

This application claims the benefit of U.S. Provisional Application No. 61/359,996, filed Jun. 30, 2010, the disclosure of which is incorporated herein by reference.

### BACKGROUND OF THE INVENTION

This invention relates in general to orthopedic bone drills. In performing surgical procedures involving the repair of bones, the setting of broken bones or the replacement of joints, it is frequently necessary to drill cavities or apertures suitable for receiving bone screws. For example, in the case of fractures, the screws may be utilized for securing bone plates for fixation of the fractured sections. Screws may also be used in a variety of joint replacements and compression hip plates among other applications. An example of a prior art bone drill is shown in U.S. Pat. No. 6,312,432.

### SUMMARY OF THE INVENTION

This invention relates to a drill suitable for drilling cavities or apertures in bones during the performance of surgical procedures.

Various aspects of this invention will become apparent to those skilled in the art from the following detailed description of the preferred embodiment, when read in light of the accompanying drawings.

### BRIEF DESCRIPTION OF THE DRAWINGS

FIG. 1 is an elevational side view of a drill of one embodiment of the invention.

FIG. 2 is a cross section taken along line A-A of FIG. 1.

FIG. 3 is a cross section taken along line B-B of FIG. 1.

FIG. 4 is an enlarged profile elevational view of the point end of the drill of FIG. 1.

FIG. 5 is an elevational end view of the point end of the drill of FIG. 1.

### DETAILED DESCRIPTION OF THE PREFERRED EMBODIMENT

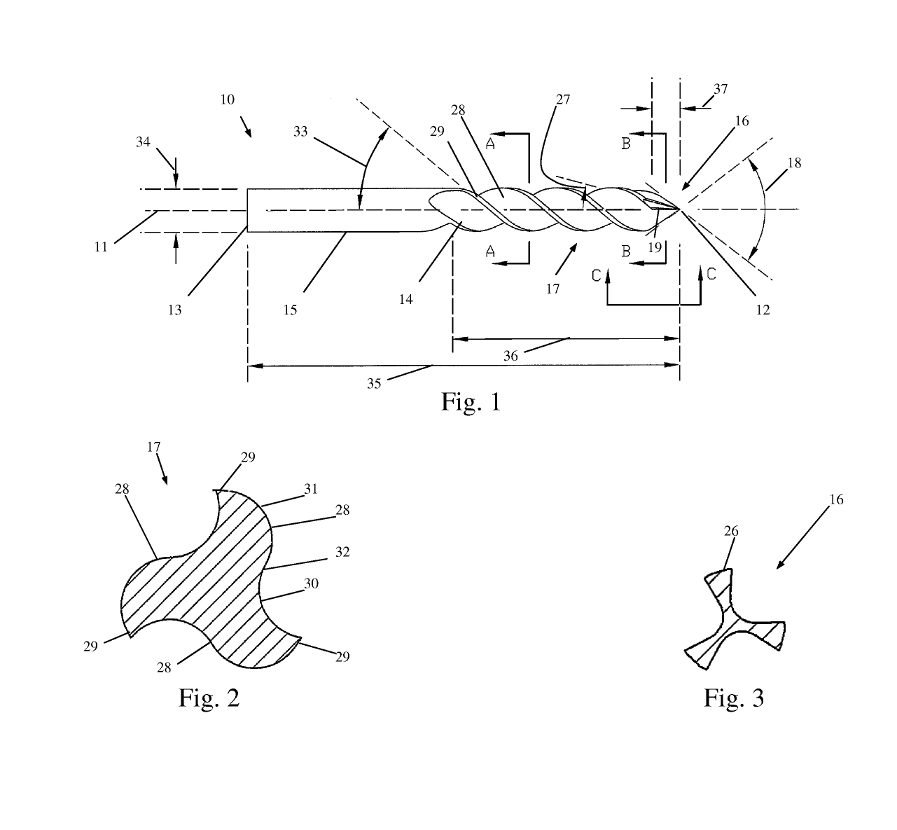

Referring now to the drawings, there is illustrated in FIG. 1 a drill bit 10 conforming to one embodiment of this invention. The drill bit 10 extends along an axis 11 from a point end 12 to a shank end 13 and includes a flute 14 and a shank 15. The drill bit 10 is manufactured of stainless steel, but this is not required, and the drill bit 10 can be manufactured of any durable material which is biocompatible.

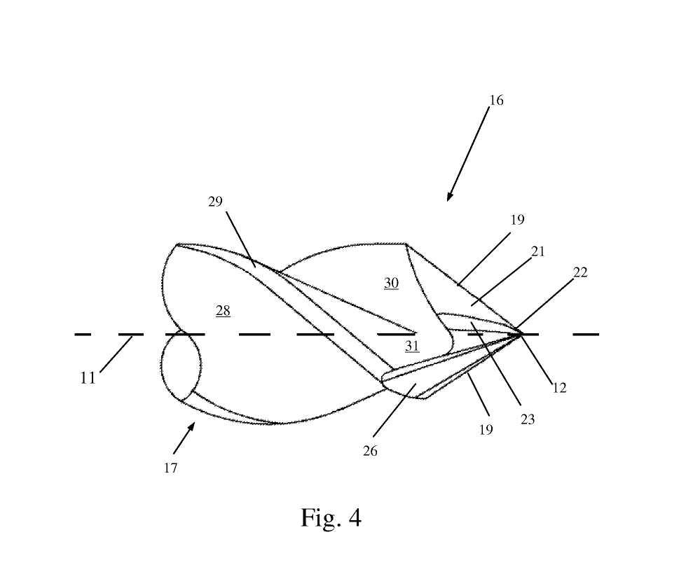

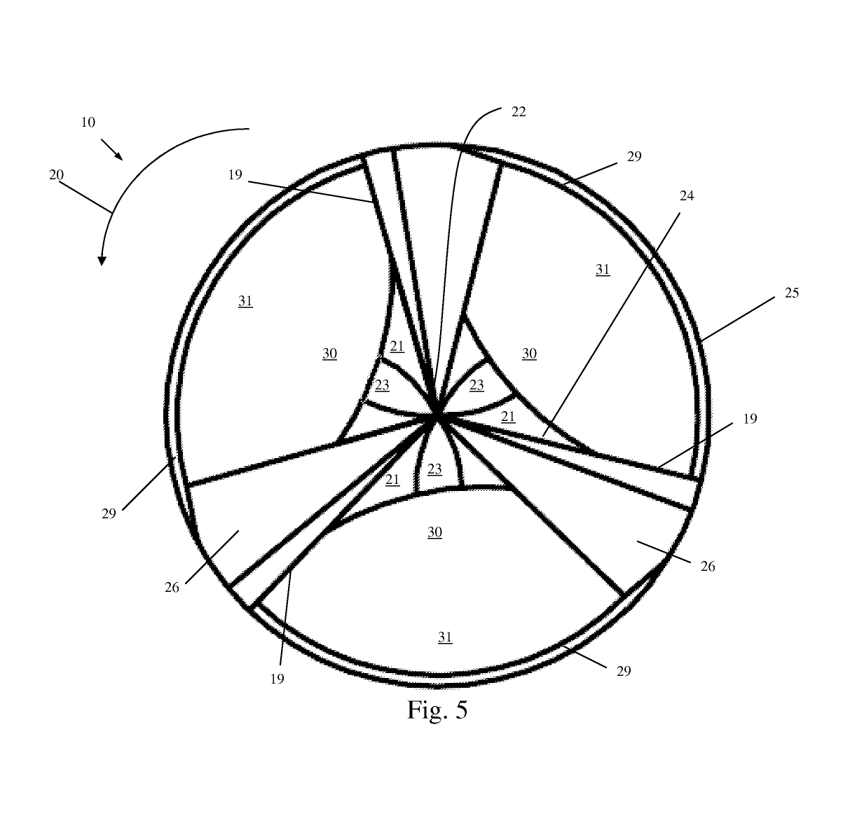

The flute 14 is the portion of the drill bit 10 that has edges for cutting and grooves for removing chips. The flute 14 includes a first helical portion or point helical portion, indicated generally at 16, and a second helical portion, indicated generally at 17. The point helical portion 16 is located nearest the point end 12 of drill bit 10. In the embodiment shown, the axial length of the point helical portion 16 is much smaller than the axial length of the second helical portion 17. The second helical portion 17 is located between the point helical portion 16 and the shank 15. As will be explained below, the point helical portion 16 may be formed differently than the second helical portion 17, such as, for example, having a different helix angle. The point end 12 may be formed by a cutting operation on the right-hand end of the first helical portion 16, as viewed in FIGS. 1 and 4.

The point end 12 is the first part of the drill bit 10 to contact a bone when the drill bit 10 is used. A point angle of a drill bit is the angle between the cutting edges projected onto a plane parallel to the axis of the drill. In the illustrated embodiment, drill bit 10 has a point angle 18 of approximately 75 degrees. This is not required, and the point angle could have a different value. The point angle 18 could be within the range of twenty degrees to ninety degrees, and is preferably within the range of sixty degrees to ninety degrees.

In the illustrated embodiment, the point helical portion 16 defines three cutting lips 19. The three first cutting lips 19 are best shown in FIG. 5. Drill bit 10 rotates counter-clockwise as viewed in FIG. 5, as shown by arrow 20. Each first cutting lip 19 is defined by a first flute 21. Point helical portion 16 also defines three second cutting lips 22. Each second cutting lip 22 is defined by a second flute 23. One second cutting lip 22 is located between each first cutting lip 19 and the point end 12. Second cutting lips 22 extend all the way to the point end 12 of drill bit 10. In the illustrated embodiment, one first cutting lip 19 and one second cutting lip 22 define a combined cutting lip 24 that is substantially continuous from the point end 12 to margin 25, that is, across the entire radius for the drill bit 10. This combined cutting lip 24 maintains the point angle 18 from the point end 12 to the margin 25. The cutting lips 19 and 22 are trailed by a web 26.

As can be seen in reference to FIG. 1, a first helix angle or point helix angle 27 defines the concave surface of the first flute 21 adjacent to each first cutting lip 19. The first flute 21 has a concave shape that is derived from the geometry of a flute grinding wheel, or a flute mill cutter, depending upon what process is utilized in the manufacture of the drill bit 10. The grinding wheel or flute mill cutter is set at a desired angle from the centerline of the drill in order to establish the point helix angle 27. In the illustrated embodiment, the point helical portion 16 has a point helix angle 27 of approximately 15 degrees. This is not required, and the point helix angle 27 could have a different value. The point helix angle 27 could be within the range of five degrees to nineteen degrees, and is preferably within the range of ten degrees to nineteen degrees. Although the illustrated embodiment is described as being manufactured using a flute grinding wheel or flute mill cutter, this is not required and drill bit 10 can be manufactured by any suitable method.

On a normal drill bit, the end of the web forms the chisel edge. As best seen in FIG. 5, it will be appreciated that drill bit 10 has no chisel edge, since the webs 26 are not joined. Rather, second flutes 23 define second cutting lips 22 and provide drill bit 10 with a cutting edge all the way to the point end 12.

Referring now to FIGS. 1 and 2, in the illustrated embodiment, second helical portion 17 defines three third flutes 28. Each of the third flutes 28 is separated by a land 29. As best shown in FIG. 2, each third flute 28 is defined by a concave portion 30 and a convex portion 31. Concave portion 30 extends from a land 29 to a flute mid-point 32. Convex portion extends from flute mid-point 32 to the next land 29. The illustrated embodiment is one possible shape of the drill bit 10, but it should be appreciated that the geometry of the second helical portion 17 can be different from that shown.

In the illustrated embodiment, second helical portion 17 has a second helix angle 33 of approximately forty degrees. This is not required, and the second helix angle 33 could have a different value. The second helix angle 33 could be within the range of thirty degrees to fifty five degrees, and is preferably within the range of thirty five degrees to forty five degrees.

Shank 15 is the portion of the drill bit 10 that has no grooves. The shank 15 includes the portion of the drill bit 10 that is held and driven by a rotational power source, such as a drill.

Dimensions of alternate embodiments of the invention are shown in tabular form below. However, dimensions other than those shown can be used in the manufacture of the drill bit 10 in accordance with the invention. As seen in reference to FIG. 1, the drill bit 10 includes an outer diameter 34, an overall length 35, an effective flute length 36, and a point length 37. The dimensions shown on the table below are in inches, unless otherwise specified.

| | Outer | Outer | | | |
|---|---|---|---|---|---|
| Outer | Diameter, | Diameter, | | | |
| Diameter, | Fraction | Decimal | Overall | Effective | Point |
| Millimeters | size | size | length | flute length | length |
| 1.2 | 3/64 | 0.0469 | 1⅜ | ½ | 0.031 |
| 2.35 | 3/32 | 0.0938 | 1¾ | ¾ | 0.061 |
| 2.8 | 7/64 | 0.1094 | 1 13/16 | 13/16 | 0.071 |
| 3.0 | — | 0.1181 | 1⅞ | ⅞ | 0.077 |
| 3.2 | ⅛ | 0.1250 | 1 15/16 | 15/16 | 0.081 |
| 3.6 | 9/64 | 0.1406 | 2 | 1 | 0.092 |
| 6.4 | ¼ | 0.2500 | 2½ | 1⅜ | 0.163 |

In the preceding disclosure, drill bit 10 is a right-handed bit. This is not required. The invention could be modified for use on a left-handed drill, if desired. Also, the preceding disclosure of drill bit 10 includes elements in sets of three. That is, there are three cutting lips 19, three third flutes 28 and so on. This is also not required. The invention could be practiced with a different number of sets of elements, if desired.

In operation, second cutting lips 22 provide that the drill bit 10 will begin cutting bone as soon as the point end 12 is in contact with bone. The steep second helix angle 33 is provided to improve the effectiveness of the bone drill 10 in removing bone chips while the drill is in use. However, a steep helix angle can lead to a corkscrew effect, causing the drill bit to actually be drawn into the bone. The shallower point helix angle 27 is provided to help avoid this corkscrewing effect. The lower point helix angle 27 of point helical portion 16 in contact with the bone being drilled helps prevent the drill bit 10 from being pulled into the bone. This allows for accurate drilling. The higher second helix angle 33 of the second helical portion 17 helps provide for removal of the bone chips formed during the use of drill bit 10.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A drill bit comprising:
a body extending along an axis and including a point end and a shank end;
a first helical portion, having a first helix angle relative to the axis; and
a second helical portion, having a second helix angle relative to the axis which is different from the first helix angle.

2. The drill bit of claim 1, wherein the first helical portion is located nearer the point end of the drill bit than the second helical portion, and wherein the second helix angle is greater than the first helix angle.

3. The drill bit of claim 2, wherein the second helix angle is between thirty degrees and fifty five degrees and the first helix angle is between five degrees and nineteen degrees.

4. The drill bit of claim 3, wherein the drill bit has a point angle between twenty degrees and ninety degrees.

5. The drill bit of claim 3, wherein the drill bit has a point angle between sixty degrees and ninety degrees.

6. The drill bit of claim 2, wherein the second helix angle is between thirty five degrees and forty degrees and the first helix angle is between ten degrees and nineteen degrees.

7. The drill bit of claim 6, wherein the drill bit has a point angle between twenty degrees and ninety degrees.

8. The drill bit of claim 6, wherein the drill bit has a point angle between sixty degrees and ninety degrees.

9. The drill bit of claim 2, wherein the second helix angle is approximately forty degrees and the first helix angle is approximately fifteen degrees.

10. The drill bit of claim 9, wherein the drill bit has a point angle between twenty degrees and ninety degrees.

11. The drill bit of claim 9, wherein the drill bit has a point angle between sixty degrees and ninety degrees.

12. A drill bit comprising:
a body extending along an axis and including a point end and a shank end; and
at least one cutting lip defined on the point end, wherein the cutting lip is substantially continuous from the outer edge of the body to the point end.

13. The drill bit of claim 12 wherein the cutting lip defines a point angle and wherein the point angle is substantially continuous throughout the cutting lip.

14. The drill bit of claim 13, further comprising a first helical portion, having a first helix angle relative to the axis, and a second helical portion, having a second helix angle relative to the axis which is different from the first helix angle, wherein the first helical portion is located nearer the point end of the drill bit than the second helical portion, and wherein the second helix angle is greater than the first helix angle.

15. The drill bit of claim 14, wherein the second helix angle is between thirty degrees and fifty five degrees and the first helix angle is between five degrees and nineteen degrees.

16. The drill bit of claim 15, wherein the drill bit has a point angle between twenty degrees and ninety degrees.

17. The drill bit of claim 15, wherein the drill bit has a point angle between sixty degrees and ninety degrees.

18. The drill bit of claim 14, wherein the second helix angle is between thirty five degrees and forty degrees and the first helix angle is between ten degrees and nineteen degrees.

19. The drill bit of claim 18, wherein the drill bit has a point angle between twenty degrees and ninety degrees.

20. The drill bit of claim 18, wherein the drill bit has a point angle between sixty degrees and ninety degrees.
